(19) United States
(12) Patent Application Publication
(10) Pub. No.: US 2007/0098650 A1
(43) Pub. Date: May 3, 2007

(54) Dental formulation

(75) Inventors: Robert Craig Miller, Short Hills, NJ (US); John Scott Karolchyk, Newton, NJ (US); Bernard Covalesky, Randolph, NJ (US)

Correspondence Address: SILLS CUMMIS EPSTEIN & GROSS P.C., ONE RIVERFRONT PLAZA, IP DEPARTMENT, NEWARK, NJ, 07102, US

(73) Assignee: Dental Creations LLC

(21) Appl. No.: 11/264,429

(22) Filed: Oct. 31, 2005

(51) Int. Cl. A61K 8/96

(52) U.S. Cl. 424/50; 424/53

(57) ABSTRACT

An orally absorbable improved dental formulation is provided. The dental formulation, which reduce the occurrence of oral plaque, reduce gingival inflammation and bleeding, and whiten the user's teeth, may be a toothpaste or a rinse which comprises the following active ingredients: carbamide peroxide, papain, xylitol, and coenzyme Q10 (ubiquinone)

### RELATED APPLICATIONS

This application claims the benefit of U.S. Provisional Application No. 60/324,317, filed Nov. 2, 2004.

### FIELD OF THE INVENTION

The present invention relates to an oral formulation for enhancing a user's immune response to gum disease and tooth decay, and which at the same time bleaches and whitens the user's teeth.

### BACKGROUND OF THE INVENTION

Oral hygiene is considered to be the best means of prevention of cavities (dental caries), gingivitis, periodontitis, and other dental disorders. It also helps to prevent bad breath (halitosis). Oral hygiene is necessary for all persons to maintain the health of their teeth and mouth.

Oral hygiene compositions fall into two main categories: (1) dentifrices and (2) mouthwashes and rinses. The most common dentifrice, toothpaste, typically consists of a mild abrasive dispersed in a gel or paste base, with detergents added to aid in cleaning, and occasionally with fluoride added to reduce tooth decay. Oral (mouth) rinses are also used frequently as part of an oral hygiene regimen.

Toothpaste is an essential part of effective oral hygiene. Toothpaste typically performs several functions that promote good oral hygiene: it can aid in rubbing dental plaque and food off of the teeth, it can eliminate or mask undesirable breath odors, and it can deliver active ingredients such as fluoride to prevent disease of the teeth and gums.

Oral rinse and mouthwash compositions have also been used for many years for the prevention of bad breath and for the elimination of bacteria and other oral microorganisms that are responsible not only for bad breath but also tooth decay, plaque and gum disease such as gingivitis and periodontitis. To this end mouthwashes in the past have been designed to clean the oral cavity, provide fresh breath and kill the harmful bacteria.

Nutritional supplements have been in use for less than a century and typically are supplied in forms to be swallowed and digested for subsequent dispersal throughout the body. Over the past one hundred and fifty years certain medications have been formulated to be absorbed directly through the mucus membranes of the mouth. Building on the evidence of high absorbability of medications through the lining of the mouth, nutritional supplements are also now recognized to be able to be absorbed in this manner and can thus be incorporated into dentifrices and oral rinses. Further, homeopathic remedies have, since their inception, been routinely administered orally.

Whitening agents in toothpastes are a relatively recent development. Originally, whitening agents were comprised of abrasives, which did not actually whiten or bleach, but simply cleaned the teeth. More recently, hydrogen peroxide and carbamide peroxide have been used to bleach and whiten teeth.

### SUMMARY OF THE INVENTION

The present invention is directed to an orally absorbable improved dentifrice in the form of a toothpaste or oral/dental rinse. More specifically, the invention relates to a toothpaste and oral rinse formulation which reduce the occurrence of oral plaque, reduce gingival inflammation and bleeding, and whiten the user's teeth.

The dental formulation of the present invention includes a base to which an active component is added. The active component includes one or more ingredients for physiologically and/or chemically reacting with the teeth and/or gums of a user. Particularly, the active component comprises carbamide peroxide in an amount between approximately 1 and 50 weight percent, coenzyme Q10 (also known as ubiquinone and commonly abbreviated “CoQ10”) in an amount between 0.01 and 10 weight percent, papain in an amount between approximately 0.1 and 10 weight percent, and xylitol in an amount between 1 and 40 weight percent.

Coenzyme Q10 has received a great deal of attention over the last 20 years. Researchers have used this agent to treat various cardiac disorders such as ischemic heart disease, congestive heart failure, toxin induced cardiomyopathy, angina pectoris, hypertension, and for gingivitis and periodontal disease. It is theorized that coenzyme Q10 acts as a cellular energy catalyst and stimulates the immune system.

Coenzyme QIO exists naturally in the mitochondria of all cells in the human body, and has indispensable functions in the bioenergetics of human tissues, including the gingiva. A deficiency of CoQ10 has been found in the gingiva of patients with periodontal disease. Many clinical trials with oral administration of CoQ10 to patients with periodontal disease have been conducted and the results have shown that oral administration of CoQ10 increases the concentration of CoQ10 in the diseased gingiva and effectively suppresses advanced periodontal inflammation. (Hanioka, et al., Molecular Aspects of Medicine 15: 1994; s241-s248).

In one form, the dental formulation of the invention consists of a toothpaste composition, which boosts the user's immune response to gum disease and tooth decay in addition to cleaning the user's teeth, bleaching the user's teeth, and freshening the user's breath. The inventive formulation may be used in conjunction with a mouthwash rinse impregnated dental floss, or chewing gum.

Accordingly, the invention provides an improved oral dental composition for enhancing a user's immune response to gum disease and tooth decay.

Also provided is a dental formulation which provides an enzymatically active dental composition and which is in the form of a biologically absorbable dental composition.

Also provided is a formulation which is safe for the user and which whitens and bleaches the user's teeth.

The invention accordingly comprises the compositions embodying the features and construction, combination of elements and component parts as exemplified in the detailed disclosure hereinafter set forth.

### DETAILED DESCRIPTION OF THE INVENTION

A. Definitions

Unless defined otherwise, the scientific and technological terms and nomenclature used herein have the same meaning as commonly understood by a person of ordinary skill to which this invention pertains.

As used herein, “hydrolyzed silica gel” is intended to refer to a micronized form of silica that is water absorbable and therefore gels and thickens. Typically, silica gel occurs as a fine, amorphous powder that is insoluble in water and most acids. Silica gel has great adsorptive power and is used as a drying agent, as a catalyst or catalyst carrier, and in purifying various substances. Silica gel is used as a flow-aid, thickener, and for other purposes in pharmaceuticals.

As used herein, “sodium lauryl sulfate” refers to a foaming agent naturally derived from coconut oil.

As used herein, “xylitol” refers to a naturally occurring sweetener substance that can be found in plants, fruits, and vegetables and is even produced in the human body by normal metabolism. Xylitol has no known toxicity or carcinogenicity. It is listed by the U.S. Food and Drug Administration as an ingredient that is Generally Recognized As Safe (GRAS).

As used herein, “glycerin” refers to an emollient or humectant that helps to balance or maintain moisture levels. It is commonly derived from oils or created as a by-product of soap. These oils can be vegetable, animal, or petroleum in origin.

As used herein, “stevia” and “stevia powder” is intended to refer to a sweetener that is extracted from an herb and contains almost no caloric value.

As used herein, “papain” and “papain powder” is intended to refer to a protein-cleaving enzyme derived from papaya and certain other plants.

As used herein, “carbopol” is intended to refer to a carbomer used mainly in liquid or semisolid pharmaceutical formulations as gelling, suspending or viscosity-increasing agents. Carbopol is also used to stabilize, suspend, and control the release of pharmaceutical products. Carbopol is a registered trademark of Noveon, Inc., Cleveland, Ohio.

As used herein, “carbamide peroxide” is intended to refer to an oxidizing agent, consisting of hydrogen peroxide compounded with urea.

As used herein, “trolamine” is intended to refer to triethanolamine, a widely used pharmaceutical emulsifying agent that also plays a role in pH adjustment.

The inventive dental formulation, exclusive of the base to which it is added, includes based on the overall weight of the active component, Coenzyme Q10 (2,3 dimethyl-5 methyl-6-decaprenyl benzoquinone; C₅₉H₉₀O₄), in an amount between about 1 and 10 weight percent.

Coenzyme Q10 is an endogenous antioxidant found in small amounts in meats and seafood. Although CoQ10 is found in all human cells, its highest concentrations occur in the heart, liver, kidneys, and pancreas. Added to the inventive formulation, Coenzyme Q10 will enhance the health of the gums and is effective for treating periodontal disease.

In addition to ubiquinone, another active ingredient is carbamide peroxide (CO(NH₂)₂.H₂O₂), in an amount between about 0.01 and 10 weight percent. Carbamide peroxide works by breaking down into water and oxygen via radical intermediates. It is believed that these radical intermediates react with polyphenols and other pigments that stain teeth, at least by destroying the double-bond network that lends such compounds their color. Significant teeth shade lightening is thus achieved. It is to be understood that the invention is not limited to carbamide peroxide and other oxidizing agents such as, but not limited to hydrogen peroxide, calcium peroxide, sodium bicarbonate and natural fruit enzymes, could be used without detracting from the invention.

Another active ingredient in the formulation is xylitol (C₅H₁₂O₅), in an amount between about 1 and 40 weight percent. Xylitol is a sugar alcohol, or polyol. Xylitol metabolism does not require insulin and does not promote tooth decay. Products sweetened with xylitol can be labeled “sugar free.” Additionally, since xylitol is metabolized independently of insulin, it can be used safely as sweetener for diabetics. Xylitol extracted from birch tree pulp is used in candies, chewing gum, and natural-ingredient toothpastes. Xylitol also demonstrates antigingivitis and remineralization of enamel properties, and has been shown to effectively reduce the prevalence of mutans streptococci which leads to dental caries. This ingredient also facilitates calcium movement and accessibility.

In toothpastes, xylitol is a multifunctional ingredient, and has several applications. For example, in addition to its sweetening properties, xylitol has cool, soothing properties that prevent “dry mouth”. It is also used as an antiplaque and enhances certain flavorings used in the toothpaste formulation. It is to be understood that the invention is not limited to xylitol and other sweetening agents such as, but not limited to sodium saccharin, cyclamates, dextrose and sorbital could be used without detracting from the invention.

Papain, a proteolytic enzyme derived from papaya and certain other plants, and which demonstrates significant non-abrasive removal of tooth deposits, may also be added to the active component of the inventive formulation in an amount between about 0.1 and 10 weight percent. Papain has also been shown to have a tooth whitening effect as well as some antibacterial properties. While papain is preferred, other enzymes, such as calcium glycerophosphate, glucose oxidase, lactoperoxidase, are contemplated as well.

One or more flavor components may be used in the practice of the present invention. In one embodiment of the present invention spearmint in a water solution is used as a flavoring. The invention is not, however, limited in the flavor components used and other types of mints, oils and flavorings are contemplated.

The toothpaste formulation of the present invention also includes one or more gelling agents. Although the present invention utilizes Carbopol 940, other gelling agents known in the art may be used without detracting from the inventive aspects of the present invention.

While some oral hygiene products presently on the market address the need for cleaning the teeth and administering fluoride, no existing product takes full advantage of the ability of such a product to deliver to the oral cavity an array of beneficial ingredients in one product, such as ubiquinone, carbamide peroxide, papain and xylitol. Furthermore, the formulation described herein is directly absorbable through the mouth and teeth without need of assimilation through the digestive system.

Additionally, the formulation described herein may be incorporated into dental carriers in common use, such as toothpaste, mouthwash, impregnated dental floss or chewing gum, so that individuals can gain the advantage of use without the need for taking a pill. The formulation may also be incorporated into carriers commonly used in the environment of the dental office, carriers such as dental prophylaxis paste, oral subgingival irrigation fluid, or biologically absorbable or nonresorbable fiber matrices, so that the benefits of these key, orally absorbable nutrients, cleaning effects, bleaching effects, favorable taste, and immune system stimulators can become part of the existing armamentarium of dentists and dental hygienists.

Toothpaste Formulation

The toothpaste in accordance with the present invention is comprised of a base and an active paste or gel.

In manufacturing 100 g of the base, the ingredients and amounts are shown in the Chart 1.

CHART 1

| Ingredient | Quantity | |
|---|---|---|
| Hydrolyzed Silica Gel | 1.5 | g |
| Sodium Lauryl Sulfate NF | 1.0 | g |
| Spearmint - water solution | 6 | ml |
| Stevia Powder Extract | 1 | g |
| Carbopol 940 (3.5% gel) | 22.5 | g |
| Glycerin USP (Natural) | 9 | g |
| Water (distilled) | 100 | g |
| Papain powder (purified) | 1 | g |
| Xylitol (D) NF | 10 | g |
| Coenzyme Q10 | 0.1 | g |

The ingredients in the manufacture of 100 g of carbamide peroxide active paste or gel are set forth in Chart 2.

CHART 2

| Ingredient | Quantity |
|---|---|
| Carbamide Peroxide | 30 g |
| Carbopol 940 (3.5% gel) | 100 g |
| Water (sterile for irrigation) | 14.2875 ml |
| Trolamine NF (Triethanolamine) | 0 gtts (to adjust pH = 7) |

Mouthrinse Formulation

The mouthrinse in accordance with the present invention is comprised of the ingredient (for 100 ml quantity) set out in Chart 4.

CHART 4

| Ingredient | Quantity | |
|---|---|---|
| Ethanol 95% | 12.5 | ml |
| Glycerin (USP) natural | 12.5 | ml |
| Spearmint - water solution | 2.0833 | ml |
| Stevia Powder Extract | 0.5 | g |
| Carbamide Peroxide | 1 | g |
| Papain Powder (purified) | 1 | g |
| Xylitol (D) NF | 10 | g |
| Coenzyme Q10 | 0.1 | g |
| Water (distilled) | 100 | ml |

In one embodiment of the present invention, the toothpaste formulation is contained in a tube or container having two chambers which are typically separated. In this manner, the base and active paste or gel are dispensed from the tube or container separately onto a toothbrush and may give the appearance of being layered. In another embodiment, the base and the active paste or gel may be contained in a tube or container and formulated to be dispensed from a single chamber.

The rinse formulation according to the present invention is intended for rinsing the mouth, teeth and gums and then expelling the rinse from the user's mouth. The rinse may be used alone or in combination with the brushing of one's teeth.

In one embodiment, the formulation of the invention has the following concentrations of active ingredients:

Toothpaste

| | |
|---|---|
| Coenzyme Q10 | 1% |
| Carbamide Peroxide | 15% (final concentration when mixed 50/50 with |
| | base) |
| Xylitol | 10% |
| Papain | 1% |

Dental Rinse

| | |
|---|---|
| Coenzyme Q10 | 0.1% |
| Carbamide Peroxide | 1-3% |
| Xylitol | 10% |
| Papain | 1% |

In accordance with the present invention, the amounts of ingredients are not limited to the above-described amounts and percentages. It is contemplated that the active ingredients may be present in the formulations in the following ranges:

| | |
|---|---|
| Carbamide peroxide - | 1% to 50%; |
| Coenzyme Q10 - | 0.01% to 10%; |
| Papain - | 0.1% to 10%; |
| Xylitol - | 1% to 40% |

In addition to the above-described essential components, the composition of the present invention can further contain additional ingredients and vehicles generally used for preparations for use in the oral cavity, such as toothpastes and dental rinses. These optional components include, but are not limited to, such components as abrasives, surfactants, thickening agents, buffers, humectants, preservatives, and anti-caries agents. All of these additives, described in further detail below, are generally usual and would be known to one of skill in the art.

In other embodiments, dental abrasives may be useful in the dentifrice compositions of the present invention. The abrasives may include a variety of different materials known in the art and preferably one which is compatible with the composition of interest and which does not excessively abrade dentin. Suitable abrasives include for example, silica precipitates; insoluble polymetaphosphate, hydrated alumina, resinous abrasives such as polymerized resins (e.g. ureas, melamines, cross-linked epoxides, phenolics, and the like), and mixtures thereof.

Another component of the dentifrice compositions of the present invention is a humectant. The humectant serves to keep compositions such as toothpaste compositions from hardening upon exposure to air, and to give mouthwash and toothpaste compositions a moist feel to the mouth. Certain humectants can also impart desirable sweetness of flavor to toothpaste and mouthwash compositions. While the formulation of the present invention utilizes glycerin, other suitable humectants for use in compositions of the present invention include edible polyhydric alcohols such as sorbitol, polyethylene glycol, and propylene glycol.

The dentifrice compositions of the present invention can also contain additional sweeteners such as saccharin sodium, acesulfame potassium, glycyrrhizin, perillartine, thaumatin, cyclamates, dextrose and aspartylphenylalanyl methyl ester.

Buffering agents may also be added to the dental compositions of the present invention. The buffering agents serve to retain the pH of the compositions within the preferred range.

Other optional components of the dentifrice compositions of the present invention are preservatives, such as those that prevent microbial growth in the dentifrice compositions. Suitable preservatives include but are not limited to methylparaben, propylparaben, benzoates, and alcohols such as ethanol.

Additional binders and thickeners can also be used in the dentifrice compositions of the present invention, particularly in toothpaste compositions. Preferred binders and thickening agents include, but are not limited to, carrageenan (e.g. Viscarin, Irish moss, and the like); cellulose derivatives such as hydroxyethyl cellulose, sodium carboxymethyl cellulose, and sodium carboxymethyl hydroxypropyl cellulose, carboxyvinyl polymers; natural gums such as karaya gum, gum Arabic, acacia and tragacanth; polysaccharide gums such as xanthan gum; filmed silica; and colloidal magnesium aluminum silicate.

Compositions of the present invention may also contain one or more surfactants. Suitable surfactants are those which are reasonably stable and preferably form suds (bubbles) throughout the pH range of the dentifrice compositions. Surfactants can also be added to act as solubilizing agents to help retain sparingly soluble components in solutions or mixtures. Surfactants useful in the dentifrice compositions as sudsing agents can be soaps, polysorbates, poloxamers, and synthetic detergents that are anionic, nonionic, cationic, zwitterionic, or amphoteric, and mixtures thereof.

Nutrients and vitamins can also optionally be added to the compositions of the present invention. Such agents can include folates, retinoids (Vitamin A), Vitamin B (B₁-thyamin, B₂-riboflavin, B₃-niacine, B₅-pantothenic acid, B₆-pyridoxine, B₇-biotin, B₈/B₉/Bc-folic acid, B₁₂-cyanocobalamin), Vitamin C (ascorbic acid, sodium ascorbate), Vitamin E, Vitamin E analogs (dl-α-tocopherol acetate, tocopherol succinate, tocopherol nicotinate) and zinc.

A variety of miscellaneous additives can also be optionally formulated into the formulations of the present invention, such as tooth desensitizing agents (e.g. potassium and strontium salts), condensed anti-tartar agents such as sodium and potassium tetrapyrophosphate, whitening agents such as aluminum oxide and calcium peroxide, debriding agents such as sodium bicarbonate, pigments and dyes, such as Blue 15-C174160, Green 7-C174260, Reds 4-CI12085 and 40 CI16035, Yellows 115 CI47005:1 and 5 CI19140, and Carmine 5 CI16035), as well as additives such as mica and sparkles. As with the other optional dentifrice additives, use can be made of either one of these ingredients or a mixture of two or more of these ingredients in amounts appropriate for the dentifrice composition.

The formulation of the present invention may also include one or more anti-caries agents, anti-plaque agents, anti-tartar agents, and anti-inflammatory agents as would be known to one of skill in the art.

With specific regard to dental rinses, various other materials may be incorporated into the mouthwashes of the present invention which will be well known to those skilled in the art. These include, for example, additional sweeteners; preservatives such as sodium benzoate and parabens; fluoride ion sources such as sodium fluoride, sodium monofluorophosphate and amine fluorides; anti-tartar agents such as water-soluble pyrophosphate salts, preferably alkali metal pyrophosphates; agents which reduce tooth sensitivity; coloring components, and vitamins, nutrients or other natural components.

Suitable mouthwash compositions according to the invention are compositions which may require shaking prior to use but which provide stable suspensions during use.

A typical oral hygiene regimen using the products of the present invention will include the steps of brushing one's teeth using the toothpaste formulation described herein by applying a dab of each of the active paste and the base on a toothbrush. After brushing teeth for approximately 1 minute or more, the oral rinse formula is swirled around in the user's mouth so as to contact all the teeth. Typically, the oral rinse will be swirled in the user's mouth for approximately 30 seconds and then expectorated. A typical oral hygiene regimen using the toothpaste and oral rinse of the present invention would involve at least 2 uses each day—once in the morning and once in the evening.

Repeated use of the toothpaste and oral rinse of the present invention will result in whiter, brighter teeth. Additionally, the toothpaste and oral rinse will reduce the occurrence of oral plaque on the user's teeth as well as reduce gingival inflammation and bleeding.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

General Manufacturing Instructions for Toothpaste:

The toothpaste consists of two final preparations—the active paste and the base paste.

Active Paste: (To prepare 100 grams) 3.5 g of carbopol 940 is sprinkled into 60 mL cold water in a beaker with a motor-driven propeller-type agitation. Once uniform, sodium hydroxide 20% solution is added with mixing until the final pH of gel is 6.5. 30 g of carbamide peroxide and 10 mL of flavor is then added to gel. The gel is then transferred to bowl of vacuum mixer and the unit is sealed with a cover. 28 inches or more of vacuum is applied for 30-45 minutes with continuous agitation.

Base Paste: (To prepare 100 grams) 9 g of glycerin and 30 mL of water are added in a beaker with motor-driven propeller-type agitation. Once uniform, 0.8 g of carbopol 940 is sprinkled into solution with agitation. Once uniform, 1.5 g of hydrophilic silica gel and 10 g of xylitol is added with agitation. Once uniform, 1 g of stevia, 6 mL of flavor and 1 g of papain are added. This mixture is then transferred to the bowl of a vacuum mixer. 0.1 g of CoQ10 and 0.75 g of sodium lauryl sulfate are then added with agitation. Once completely uniform, the unit is sealed with a cover 28 or more inches of vacuum are applied for 30-45 minutes with continuous agitation.

Successful toothpaste preparation, even in the laboratory, requires a means of excluding or removing entrapped air. This is best accomplished by conducting the entire manufacturing operation under vacuum. Where vacuum equipment is unavailable, or as a supplement, a unit called a Versator may be employed. However, a system found to be useful in the laboratory setting is to enclose an electric mixer in a stainless steel tank fitted with an airtight cover and connected to a vacuum pump.

Cold method preparation: The paste is prepared as follows. The humectant, such as glycerin, is added to the bowl of the mixer. Binder is sprinkled in under agitation so that particles are dispersed in the absence of water, preventing swelling at this point. A separate liquid phase is prepared, which includes the available water, sweetener, preservatives and any therapeutic additives. This solution is added to the humectant/binder mixture. The mixture is placed under vacuum for 10 minutes to de-aerate the thick gelatinous liquid phase. Although the formulation of the present invention does not include abrasives, in alternative embodiments in which abrasives may be added, the vacuum is opened at this point and the abrasives are added with mixing until they are thoroughly wet down. Vacuum is reapplied and the paste is mixed for at least 30 minutes under 28 inches or more of vacuum. In the meantime, the surface active agents and flavors are dispersed in about 5% of the available humectant. At the conclusion of the 30 minute time, the vacuum is again opened and the flavor mixture is added. Five minutes of additional mixing under vacuum will produce a smooth, air free paste.

### Example 2

General Manufacturing Instructions for Oral Rinse:

Cold Preparation Technique: (To prepare 100 mL) In a calibrated vessel, 12.5 mL of ethanol 95%, 0.5 mL of polysorbate 80, 12.5 mL of glycerin and 50% of available water are added and mixed until uniform. Next 0.5 mL of stevia, 10 g of xylitol and 2.5 mL of flavor are added and mix until uniform. Next, the active ingredients, 1 g of carbamide peroxide, 0.1 g of CoQ10 and 1 g of papain, are added and mixed under high agitation until completely uniform.

### Example 3

Testing Efficacy of Formulation

A 6-week study will be conducted in 32 subjects to evaluate the effects of an oral hygiene system on plaque, gingivitis and tooth whitening. Subjects selected for the study will present at screening with moderate plaque, American Academy of Periodontology (AAP) Type 1/11 periodontal status. Evaluation data will be collected at baseline and days 14, 28 and 42. The Plaque Index (PI) of Silness and Loe will be used to quantify the amount of supragingival plaque surrounding six selected teeth (7, 8, 9, 10, 24 and 25) the Gingival Index (GI) of Loe and Silness will be used to assess the bleeding tendencies and visual appearance on the same six teeth. The VitaShade index will be used to evaluate tooth color on the same six teeth. Additionally, Chromameter® (Minolta) measurements will be taken to measure the color of the selected teeth. Subjects will be asked to perform study treatment 2 times a day, after meals. Subjects will be asked to complete a diary to assess compliance. Data will be analyzed within each mouth location and product.

### Example 4

Test Procedure

The study is designed as a 6-week study in which the test system will be used by each of the test panelists according to the Sponsor's use instructions.

At the baseline visit, each potential subject will be qualified by the study Dentist for bleeding after probing at distinct sites in the mouth with a 6″ probe and will be qualified according to the gingival index. The bleeding will be assessed by the study Dentist and categorized as mild, moderate or severe and pocket depth will be recorded as well. A visual evaluation will be conducted using a 0.2% erythrosine disclosing agent to calculate plaque levels. Subjects meeting this and all other inclusion/exclusion criteria will be enrolled. Following enrollment, the study Dentist will use the Minolta Chromameter® to evaluate the color of six selected teeth (tooth numbers: 7, 8, 9, 10, 24 and 25). Additionally, a VitaShade® grade will be assigned to the six selected teeth. The study Dentist will also record any other oral conditions that are present that may impact upon the study results. Following the evaluations, the enrolled subjects will receive a test system, use instructions and a daily diary on which to record system application times and any pertinent comments. Subjects will be instructed to call the Testing Facility after 1 week of product use to report problems (if any) and to return to the Testing Facility on days 14, 28 and 42 of product use to be re-examined by the study Dentist.

Evaluations of efficacy will be based on a comparison of baseline vs. each observation period.

### Example 5

Clinical Evaluation Procedures

Evaluations for all parameters will be conducted according to the procedures outlined below.

A. Evaluation of Plaque Index

The Plaque Index (PI) of Silness and Loe will be used to quantify supragingival plaque accumulation. Plaque scores will be recorded from the same six selected teeth (7, 8, 9, 10, 24 and 25) at the same sites. Three drops of an erythrosine disclosing agent will be placed into a paper cup and gently dispersed on the teeth with a cotton tip applicator. Subjects will be asked to rinse and expel. A score of 0-3 will be assigned for each tooth using the following criteria:

| | |
|---|---|
| 0 | No plaque present. |
| 1 | Presence of a film of plaque adhering to the gingival margin |
| | and adjacent area of the tooth. |
| 2 | Presence of moderate accumulations of plaque within the |
| | gingival sulcus that can be seen with the naked eye, or on the |
| | tooth and gingival margin. |
| 3 | Presence of an abundance of plaque within the gingival |
| | sulcus and/or on the tooth and gingival margin. |

B. Evaluation of Tooth Color/Shade

At all evaluations, the study Dentist will evaluate the color/shade of the six pre-selected teeth of each subject and assign a Vita Shade® grade to each tooth. Additionally, the color/shade of the six selected teeth will be measured using the Minolta Chromameter®.

C. Evaluation of Gingival Bleeding

At each evaluation, the study Dentist will assess the Gingival Index (GI) of Loe and Silness to assess the severity of gingival tissues adjacent to selected teeth numbers 7, 8, 9, 10, 24 and 25 at distal, facial, mesial, and lingual sites. A score of 0-3 will be assigned for each tooth using the following criteria:

| | |
|---|---|
| 0 | Absence of inflammation |
| 1 | Presence of mild inflammation, slight change in color, slight |
| | edema, and no bleeding on probing |
| 2 | Presence of moderate inflammation, moderate redness and |
| | edema with bleeding on probing. |
| 3 | Presence of severe inflammation, marked redness and edema, |
| | and tendency toward spontaneous bleeding. |

D. Evaluation of Pocket Depth

At all evaluations, the study Dentist will measure the pocket depth at each test site.

E. Safety Assessment

At all evaluations the study Dentist will assess the overall condition of the teeth and gums of the subject.

F. Daily Diary/Test Product Compliance

Subjects will be asked to complete a daily diary to assess product compliance, record treatment times, and deviations from the protocol instructions, concurrent medications, and to elicit any additional comments.

It will thus be seen that the objects set forth above, among those made apparent from the preceding description, are efficiently attained, and since certain changes may be made in the above compositions, without departing from the spirit and scope of the invention, it is intended that all matter contained in the above description shall be interpreted as illustrative, and not limiting.

1. A dental formulation comprising:
(a) coenzyme Q10 in an amount of 0.1% to 1% by weight, based on the total weight of the composition;
(b) carbamide peroxide in an amount of 14% to 40% by weight, based on the total weight of the composition;
(c) xylitol in an amount of 10% to 30% by weight, based on the total weight of the composition; and
(d) papain in an amount of 1% to 5% by weight, based on the total weight of the composition.

2. The dental formulation according to claim 1, wherein said formulation is a toothpaste or an oral rinse.

3. The dental formulation according to claim 1, wherein said formulation further comprises a flavoring.

4. A toothpaste composition comprising a tooth whitening effective amount of:
(a) coenzyme Q10 in an amount of 0.1% to 1% by weight, based on the total weight of the composition;
(b) carbamide peroxide in an amount of 14% to 40% by weight, based on the total weight of the composition;
(c) xylitol in an amount of 10% to 30% by weight, based on the total weight of the composition; and
(d) papain in an amount of 1% to 5% by weight, based on the total weight of the composition.

5. The toothpaste composition according to claim 4 comprising one or more base components and an active component.

6. The toothpaste composition according to claim 45, wherein said base component comprises:
(a) hydrolized silica gel;
(b) sodium lauryl sulfate;
(c) stevia powder extract;
(d) carbopol;
(e) glycerin;
(f) water;
(g) papain;
(h) xylitol; and
(i) coenzyme Q10

7. The toothpaste composition according to claim 5, wherein said active component comprises:
(a) carbopol;
(b) carbamide peroxide;
(c) triethanolamine; and
(d) water.

8. The toothpaste composition according to claim 5, wherein said active component is a paste or gel.

9. The toothpaste composition according to claim 4, further comprising one or more therapeutic agents selected from the group consisting of: anti-caries agents, anti-plaque agents, anti-tartar agents, and anti-inflammatory agents.

10. A method for whitening teeth comprising the step of administering the toothpaste composition of claim 4 to a user in need thereof.

11. A formulation which is effective for use as a dental rinse or mouthwash and which formulation comprises:
(a) coenzyme Q10;
(b) carbamide peroxide;
(c) xylitol; and
(d) papain.

12. The formulation according to claim 11, further comprising water.

13. The formulation according to claim 11, wherein said coenzyme Q10 is present in the formulation in the range from approximately 0.01% to approximately 1%.

14. The formulation according to claim 11, wherein said carbamide peroxide is present in the range from approximately 14% to approximately 40%.

15. The formulation according to claim 11, wherein said papain is present in the range from approximately 1% to approximately 5%.

16. The formulation according to claim 11, wherein said xylitol is present in the range from approximately 10% to approximately 30%.

17. The formulation according to claim 11, further comprising a flavoring.

18. A method for whitening teeth comprising the step of administering the dental rinse of claim 11 to a user in need thereof.

19. A method for reducing the occurrence of oral plaque and reducing gingival inflammation and bleeding, said method comprising administering a therapeutically effective amount of a formulation comprising coenzyme Q10, xylitol, papain and carbamide peroxide within the oral cavity.

20. The method according to claim 19, wherein said formulation is a toothpaste or dental rinse.

21. The method according to claim 19, wherein said formulation further comprises one or more therapeutic agents selected from the group consisting of: anti-caries agents, anti-plaque agents, anti-tartar agents, and anti-inflammatory agents.

22. A method for treating periodontal disease and preventing tooth decay, said method comprising the step of applying an effective amount of a formulation to a patient's teeth, the formulation comprising.
(a) coenzyme Q10 in an amount of 0.1% to 10% by weight, based on the total weight of the composition;
(b) carbamide peroxide in an amount of 1% to 50% by weight, based on the total weight of the composition;
(c) xylitol in an amount of 1% to 40% by weight, based on the total weight of the composition;
(d) papain in an amount of 0.1% to 10% by weight, based on the total weight of the composition; and
(e) one or more therapeutic agents selected from the group consisting of: anti-caries agents, anti-plaque agents, anti-tartar agents, and anti-inflammatory agents.

23. The method according to claim 22, wherein said formulation is a toothpaste.

24. The method according to claim 22, wherein said formulation is a dental rinse.
